(19) United States
(12) Patent Application Publication
(10) Pub. No.: US 2025/0308105 A1
(43) Pub. Date: Oct. 2, 2025

(54) SYSTEMS AND METHODS FOR DISPLAYING PATIENT DATA RELATING TO CHRONIC DISEASES

(71) Applicant: Theragraph Inc., Edmontion (CA)

(72) Inventor: Brennan WALTERS, Edmonton (CA)

(21) Appl. No.: 18/862,918

(22) PCT Filed: May 15, 2023
(86) PCT No.: PCT/CA2023/050666

§ 371 Date: Nov. 4, 2024

Related U.S. Application Data
(60) Provisional application No. 63/364,666, filed on May 13, 2022.

(51) Int. Cl. G06T 11/20; G06T 11/00; G16H 10/60

(52) U.S. Cl. CPC G06T 11/206; G06T 11/001; G16H 10/60; G06T 2200/24

(57) ABSTRACT

Disclosed is a method for execution by a server that involves maintaining a framework for categorizing time series patient data into one of a plurality of possible categories useful for managing patients with IBD (Inflammatory Bowel Disease) and/or other chronic health conditions. The method also involves receiving time series patient data spanning over a total time period, and dividing the total time period into a plurality of time intervals. The method also involves, for each time interval of the plurality of time intervals, analyzing the time series patient data of the time interval using the framework to produce a category of the plurality of possible categories. The method also involves generating graphic data for a GUI (Graphic User Interface) to display, for each time interval, a visual indication of the category for the time interval. The method also involves transmitting the graphic data from which the GUI can be generated.

### CROSS-REFERENCE TO RELATED APPLICATION

This patent application claims priority to U.S. Provisional Patent Application No. 63/364,666 filed on May 13, 2022, which is hereby incorporated herein by reference in its entirety.

### FIELD OF THE DISCLOSURE

This disclosure relates to communication systems, and more particularly to communication systems for displaying and patient data relating to chronic diseases.

### BACKGROUND

Chronic health conditions can be very complex and treatment and monitoring thereof can generate large amounts of data. IBD (Inflammatory Bowel Disease) is an example of a complex, chronic medical condition. There has been enormous progress made in the management of patients with IBD over the past 20 years. An important driver of this improvement, for example, has been the introduction of biologic medications. However, use of these and other medications have increased complexity of patient management. In addition, patients themselves have had challenges in comprehending the true nature of their disease as well as its effective treatment.

In the United States, approximately 15% of IBD patients receive care in high-volume, tertiary referral hospitals. Therefore, about 85% of IBD patients are in fact treated in community setting (see, for example Binion D. Using Institutional Databases to Study Inflammatory Bowel Disease. Gastroenterol and Hepatol. 2016;12(4):256-259.) In Canada, this practice pattern also exists, with a majority of IBD patients receiving care outside of academic centres. In order to properly treat IBD patients, under conventional approaches, physicians monitor multiple patient variables over time, and make management decisions based on this. In the community, there is much less support for this. For example, only a small percentage of physicians are supported by a nurse to help with management of these complex patients.

Thus, unfortunately, many conventional approaches to managing chronic health conditions can be difficult and prone to error, because management relies on integrating data from multiple sources often over extensive periods of time. This integration is typically not remotely available in a concise manner. It is desirable to improve upon the conventional approaches by employing technology to eliminate or mitigate some or all of the aforementioned shortcomings.

### SUMMARY OF THE DISCLOSURE

Disclosed is a method for execution by a server that involves maintaining a framework for categorizing time series patient data into one of a plurality of possible categories useful for managing patients with chronic health conditions such as, for example, IBD (Inflammatory Bowel Disease). The method also involves receiving time series patient data spanning over a total time period, and dividing the total time period into a plurality of time intervals. The method also involves, for each time interval of the plurality of time intervals, analyzing the time series patient data of the time interval using the framework to produce a category of the plurality of possible categories. The method also involves generating graphic data for a GUI (Graphic User Interface) to display, for each time interval, a visual indication of the category for the time interval. The method also involves transmitting the graphic data from which the GUI can be generated.

The graphic data can be received by a client computing device and can enable the client computing device to generate the GUI. Advantageously, the categorization of the time series patient data within each time interval can make it easier for a physician or other medical professional to assess the patient data in an objective manner, for the purpose of managing patients with IBD. In this way, difficulties associated with subjective assessments as to what levels of patient variables over time are acceptable can be mitigated or avoided. Therefore, this is an improvement over the conventional approaches which is made possible by employing the technology summarized above.

Also disclosed are a non-transitory computer readable medium and a server that generally correspond to the method summarized above.

Some embodiments of the disclosure can be employed for other applications other than IBD, namely in other chronic medical diseases.

Other aspects and features of the present disclosure will become apparent, to those ordinarily skilled in the art, upon review of the following description of the various embodiments of the disclosure.

### BRIEF DESCRIPTION OF THE DRAWINGS

Embodiments will now be described with reference to the attached drawings in which:

FIG. 1 is an example user interface having a continuous representation of a patient variable over time in relation to other clinical parameters according to the prior art;

FIG. 2 is an example block diagram of a data analysis and communication system having a server coupled to a plurality of client computing devices via a network;

FIG. 3 is a flowchart of an example method of generating a user interface having a discretized representation of patient variables over time;

FIG. 4 is an example user interface having a discretized representation of patient variables over time; and

FIGS. 4A-4D show examples of a user interacting with the user interface of FIG. 4.

### DETAILED DESCRIPTION OF EMBODIMENTS

It should be understood at the outset that although illustrative implementations of one or more embodiments of the present disclosure are provided below, the disclosed systems and/or methods may be implemented using any number of techniques. The disclosure should in no way be limited to the illustrative implementations, drawings, and techniques illustrated below, including the exemplary designs and implementations illustrated and described herein, but may be modified within the scope of the appended claims along with their full scope of equivalents.

### Introduction

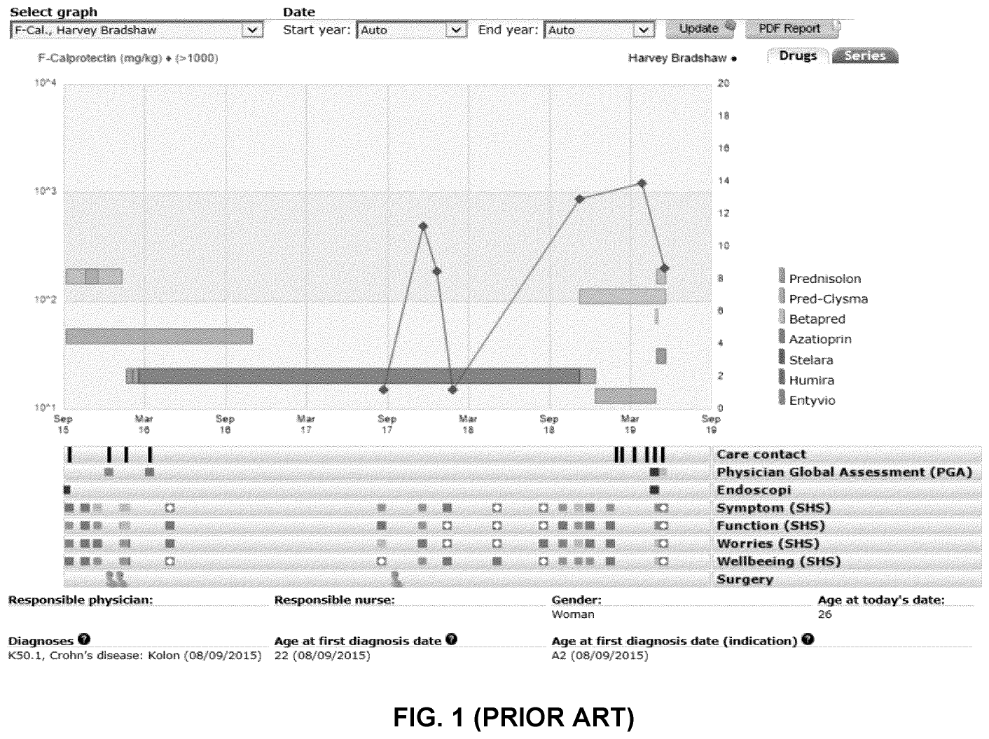

Referring first to FIG. 1, shown is a user interface having a continuous representation of a patient variable over time in relation to other clinical parameters. In the illustrated example, the patient variable is F-Calprotectin, but other patient variables are possible. A physician can monitor the single variable F-Calprotectin over time in relation to other clinical parameters, and make a diagnoses based on the patient variable and the other clinical parameters, for example whether the patient is experiencing IBD or some other ailment. However, this involves a subjective assessment as to what levels of the patient variable over time is acceptable, which can be difficult while the levels change over time. Embodiments of the disposure employ a data analysis and communication system to eliminate or mitigate some or all of the aforementioned shortcomings as described below.

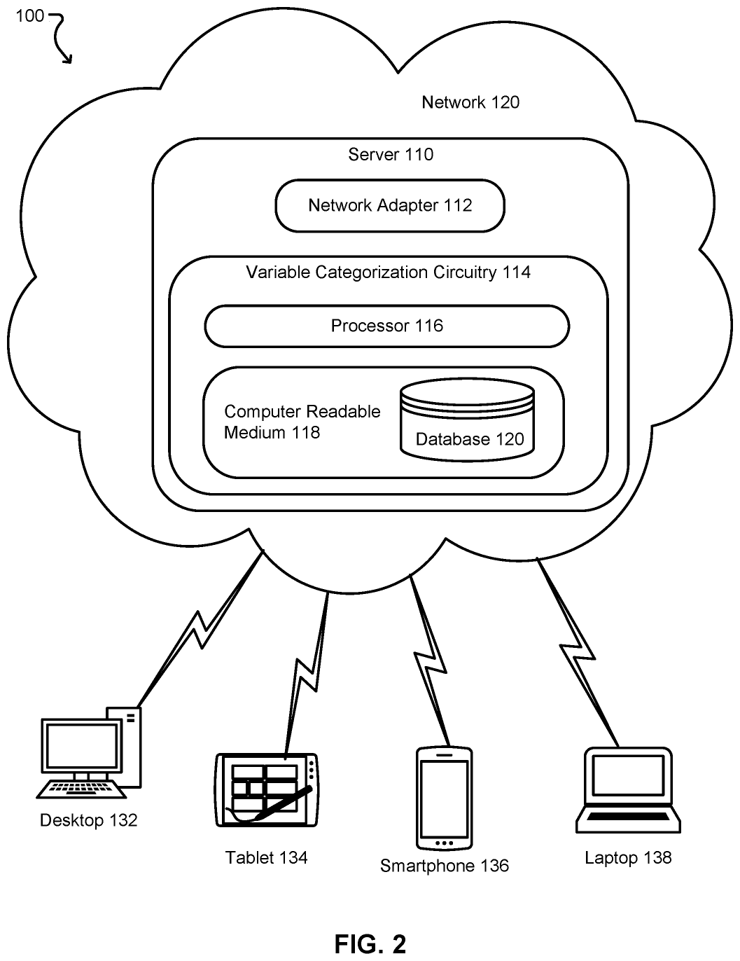

Referring now to FIG. 2, shown is a block diagram of a data analysis and communication system 100 having a server 110 coupled to a plurality of client computing devices 132,134,136,138 via a network 120. The data analysis and communication system 100 can have other components as well, but these are not shown for simplicity.

The server 110 has a network adapter 112 for communicating with the client computing devices 132,134,136,138 over the network 120. The server 110 also has variable categorization circuitry 114. The server 110 can have additional components, but these are not shown for simplicity.

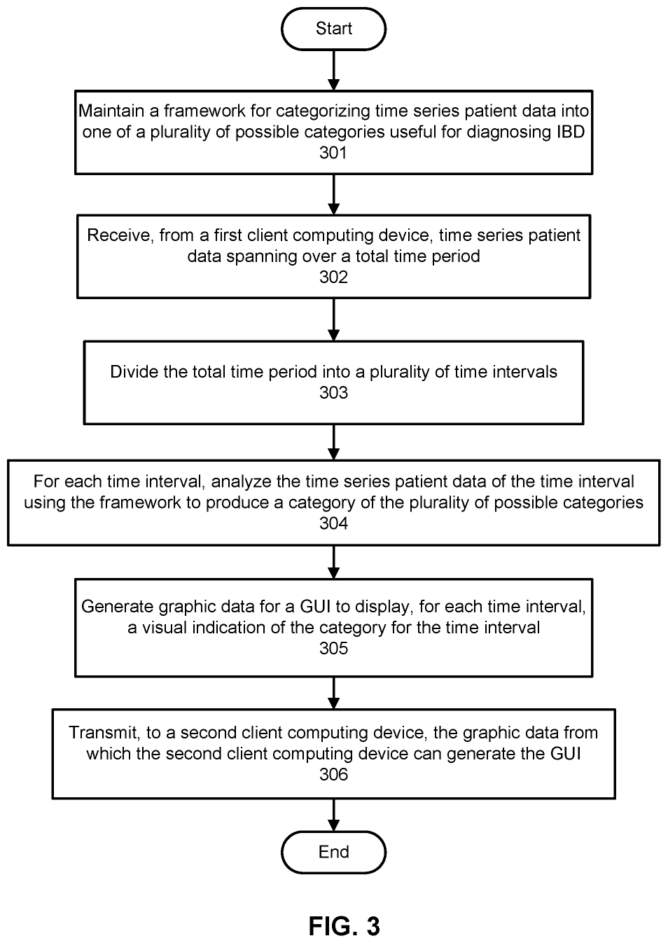

The variable categorization circuitry 114 of the server 110 operates to acquire patient data from at least some of the client computing devices 132, 134, 136, 138, and to determine and convey categorization data to at least some of the client computing devices 132,134, 136,138, based on the acquired patient data. Such operation will be described below with reference to FIG. 3, which is a flowchart of a method of determining and conveying categorization data. Although the method of FIG. 3 is described below with reference to the server 110 in the data analysis and communication system 100 shown in FIG. 2, it is to be understood that the method of FIG. 3 is applicable to other systems. In general, the method of FIG. 3 is applicable to the server 110 in any appropriately configured system.

At step 301, the server 110 maintains a framework for categorizing time series patient data into one of a plurality of possible categories useful for managing patients with IBD. In some implementations, the framework is stored in a database 120 of the server 110. However, it is noted that the framework can be stored elsewhere and even external from the server 110.

At step 302, the server 110 receives, from a first client computing device 132, time series patient data spanning over a total time period. The first client computing device 132 can be a client computing device utilized by a physician or other medical professional. In some implementations, the time series patient data is stored in the database 120 of the server 110. However, it is noted that the time series patient data can be stored elsewhere and even external from the server 110. In some implementations, step 302 may comprise the server 110 importing an electronic health record for a the patient.

At step 303, the server 110 divides the total time period into a plurality of time intervals. At step 304, the server 110, for each time interval of the plurality of time intervals, analyzes the time series patient data of the time interval using the framework to produce a category of the plurality of possible categories.

At step 305, the server 110 generates graphic data for a GUI to display, for each time interval, a visual indication of the category for the time interval. Finally, at step 306, the server 110 transmits, to a second client computing device 134, the graphic data from which the second client computing device 134 can generate the GUI. The second client computing device 134 can for example be a client computing device utilized by a physician or other medical professional. The graphic data can enable the second client computing device 134 to generate the GUI.

Advantageously, the categorization of the time series patient data within each time interval can make it easier for the physician or other medical professional to assess the patient data in an objective manner, for the purpose of managing IBD. In this way, difficulties associated with subjective assessments as to what levels of patient variables over time are acceptable can be mitigated or avoided. Therefore, this is an improvement over the conventional approaches which is made possible by employing the technology summarized above.

In some implementations, the time series patient data includes a plurality of patient variables over time, and the method includes repeating the analyzing and the generating steps for each of the patient variables. In other implementations, the time series patient data includes a single patient variable over time.

In some implementations, the plurality of possible categories consists of two categories, such for each time interval, the visual indication for each patient variable is a bimodal representation. In this way, a continuous variable is transformed into a bimodal variable. In other implementations, the plurality of possible categories includes three or more categories.

In some implementations, the framework includes a threshold function, such that a patient variable over a time interval has an average value that is compared to a threshold value to determine the bimodal representation for the patient variable in the time interval. For example, if the average value is greater than the threshold value, then the patient variable may be deemed to be too high. Conversely, if the average value is below the threshold value, then the patient variable may be deemed to be acceptable. Other frameworks are possible and are within the scope of the disclosure.

In some implementations, the bimodal representation for each patient variable comprises a first color for acceptable value of the patient variable and a second color for unacceptable value of the patient variable. In other implementations, alternative visual indications are utilized such as shading and/or hatching.

In specific implementations, the first color is blue (lighter square) and a second color is red (darker square). However, it will be appreciated that other colors are possible and are within the scope of the disclosure. Other colors can include green and yellow, for example. Some squares appear blank, which indicates that no data is available. This in itself is a very useful element of patient management.

In some implementations, the patient variables include at least some of: CDIFF (Clostridium Difficile) which is an infection of the colon that can worsen IBD, CRP (C-Reactive Protein) which is a blood test that looks at overall inflammation in the body, FCP (Fecal Calprotectin) which is a stool test that monitors inflammation in stool and provides an objective number that can be followed over time to assess response (or lack of response) to therapy, HB (Hemoglobin) which can be used to determine if someone is anemic possibly due to blood loss from an inflamed bowel, WBC (White Blood Cell) count, which is another marker for inflammation in the body, and liver enzymes (e.g. AST, ALT, ALP, Bilirubin, GGT). If any one more of these patient variables are elevated, it can be flagged as abnormal with a red square for example.

In some implementations, systems and methods according to the present disclosure can also used for displaying patient data relating to chronic conditions other than IBD. For example, in a system configured for monitoring diabetes (Type 1 and/or Type 2), the patient variables may include fasting plasma glucose, glycated hemoglobin (HbA1c)), random plasma glucose, blood pressure, lipid profile, creatinine and estimated glomerular filtration rate (eGFR), urine albumin-to-creatinine ratio, BMI, waist circumference, serum insulin levels, C-peptide levels, liver function test, thyroid function tests, vitamin D levels, hemoglobin level, microalbuminuria.

In a system configured for monitoring psoriasis, the patient variables may include psoriasis area severity index (PASI), body surface area affected (BSA), dermatology life quality index (DLQI), itch severity scale (ISS), visual analog scale for pain (VAS), nail psoriasis severity index (NAPSI), physician global assessment (PGA), patient global assessment (PtGA), Health related quality of life (HRQOL), psoriasis symptom index (PSI), joint involvement, serum drug levels, adverse events.

In a system configured for monitoring atopic dermatitis, the patient variables may include eczema area and severity index (EASI) score, scoring atopic dermatitis (SCORAD) index, investigator global assessment (IGA), patient oriented eczema measure (POEM), visual analog scale (VAS) for itch, patient global assessment (PtGA), dermatology life quality index (DLQI), CBC, liver function tests, renal function tests, serum drug levels, skin barrier function (transepidermal water loss/skin hydration), adverse events.

In a system configured for monitoring hydradenitis suppurativa, the patient variables may include hydradenitis suppurativa clinical response (HiSCR), investigator global assessment (IGA), patient global assessment (PtGA), pain, abscess, inflammatory nodules, drainage/fistulas, dermatology quality life of index (DLQI), hydradenitis suppurativa quality of life (HiSQoL), CBC, liver function tests, renal function tests, serum drug levels, adverse events.

In a system configured for monitoring rheumatoid arthritis, the patient variables may include American College of Rheumatology (ACR) response criteria, Disease Activity Score (DAS), Health Assessment Questionnaire Disability Index (HAQ-DI), C-reactive protein (CRP) level, Erythrocyte sedimentation rate (ESR), Physician's Global Assessment of Disease Activity, Patient's Global Assessment of Disease Activity, Visual Analog Scale (VAS) pain, Swollen joint count, tender joint count, Radiographic joint damage, Short Form 36 (SF-36), EuroQol-5 Dimension (EQ-5D), Fatigue, Morning stiffness duration, Patient's assessment of joint pain, Patient's assessment of joint stiffness, Rheumatoid Arthritis Quality of Life (RAQOL) questionnaire, serum levels of the biologic drug being studied, adverse events.

In a system configured for monitoring psoriatic arthritis, the patient variables may include American College of Rheumatology (ACR) response criteria, Psoriasis Area and Severity Index (PASI), Disease Activity Score (DAS), Health Assessment Questionnaire Disability Index (HAQ-DI), C-reactive protein (CRP) level, Erythrocyte sedimentation rate (ESR), Physician's Global Assessment of Disease Activity, Patient's Global Assessment of Disease Activity, Visual Analog Scale (VAS) pain, Swollen joint count, Tender joint count, Radiographic joint damage, Short Form 36 (SF-36), EuroQol-5 Dimension (EQ-5D), Fatigue, Morning stiffness duration, Patient's assessment of joint pain, Patient's assessment of joint stiffness, Psoriatic Arthritis Quality of Life (PsAQoL) questionnaire, Adverse events.

In a system configured for monitoring Ankylosing Spondylitis, the patient variables may include Assessment of SpondyloArthritis international Society (ASAS) criteria, Bath Ankylosing Spondylitis Disease Activity Index (BASDAI), Bath Ankylosing Spondylitis Functional Index (BASFI), Bath Ankylosing Spondylitis Metrology Index (BASMI), C-reactive protein (CRP) level, Erythrocyte sedimentation rate (ESR), Physician's Global Assessment of Disease Activity, Patient's Global Assessment of Disease Activity, Visual Analog Scale (VAS) pain, Spinal pain, Spinal mobility, Short Form 36 (SF-36), EuroQol-5 Dimension (EQ-5D), Fatigue, Sleep disturbances, Patient's assessment of joint pain, Patient's assessment of joint stiffness, Ankylosing Spondylitis Quality of Life (ASQoL) questionnaire, Adverse events.

In a system configured for monitoring asthma, the patient variables may include Forced expiratory volume in 1 second (FEV1), Peak expiratory flow rate (PEFR), Asthma Control Questionnaire (ACQ), Asthma Quality of Life Questionnaire (AQLQ), Asthma Symptom Utility Index (ASUI), Asthma exacerbation rate, Rescue medication use, Fractional exhaled nitric oxide (FeNO), Blood eosinophil count, Total IgE levels, Forced vital capacity (FVC), Bronchodilator reversibility, Allergen-specific IgE levels, Pulmonary function tests (PFTs, which measure lung function, including FEV1, FVC, and other parameters), Patient-reported outcomes (PROs), Inflammatory cytokine levels, Asthma exacerbation severity, Asthma symptom score, Asthma control status, which measures the level of asthma control based on established criteria, such as the Global Initiative for Asthma (GINA) guidelines, Adverse events.

In some implementations, systems and methods according to the present disclosure can be used to track multiple chronic diseases concurrently. Neither the list of diseases, nor the list of clinical variables monitored are entirely exhaustive.

In some implementations, the time intervals are eight weeks. However, it will be appreciated that other time intervals are possible and are within the scope of the disclosure. Other possible time intervals can include 6 weeks or 10 weeks, for example.

In some implementations, first client computing device 132 is different from the second client computing device 134. In other implementations, the same second client computing is involved for the receiving at step 302 and the transmitting at step 306.

There are many possibilities for the client computing devices 132, 134, 136, 138. The client computing devices 132, 134, 136, 138 can for example include a desktop computer 132, a tablet computer 134, a smartphone 136, a laptop 138, and/or any other appropriate client computing device. The client computing devices 132, 134, 136, 138 can communicate with the server 110 using wireless connections as depicted and/or wired connections. Although only four client computing devices 132,134, 136,138 are depicted, it is to be understood that there can be more or less than four computing devices.

There are many possibilities for the network 120. The network 120 can include several different networks even though such details are not shown for simplicity. For example, the network 120 can include a RAN (Radio Access Network) for communicating with wireless stations and the Internet for communicating with numerous other computing devices. The network 120 can have other components as well, but these details are not shown for simplicity.

There are many possibilities for the server 110. In some implementations, the server 110 includes a web server and the graphic data sent by the server 110 includes web content for a web browser. Additionally, or alternatively, the server 110 can include an application server and the graphic data includes content for a mobile app. Other implementations are possible.

There are many possibilities for the network adapter 112 of the server 110. In some implementations, the network adapter 112 is a single network adapter 112. In other implementations, the network adapter 112 includes multiple network adapters, for example a first network adapter for communicating with the one or more client computing devices 132,134,136,138, and a second network adapter for communicating with other client computing devices, such as client computing devices utilized by a system administrator. Both wireless and wired network adapters are possible. Any suitable network adapter that can communicate via the network 120 is possible.

There are many possibilities for the variable categorization circuitry 114 of the server 110. In some implementations, the variable categorization circuitry 114 includes a processor 116 that executes software, which can stem from a computer readable medium 118. In some implementations, the computer readable medium 118 also has a database 120 for storing the framework described above. However, other implementations, besides software implementations, are possible and are within the scope of this disclosure. It is noted that other implementations can include additional or alternative hardware components, such as any appropriately configured FPGA (Field-Programmable Gate Array), ASIC (Application-Specific Integrated Circuit), and/or microcontroller, for example. More generally, the variable categorization circuitry 114 of the server 110 can be implemented with any suitable combination of hardware, software and/or firmware.

According to another embodiment of the disclosure, there is provided a non-transitory computer readable medium having recorded thereon statements and instructions that, when executed by the processor 116 of the server 110, implement a method as described herein. The non-transitory computer readable medium can be the computer readable medium 118 of the server 110 shown in FIG. 2, or some other non-transitory computer readable medium. The non-transitory computer readable medium can for example include an SSD (Solid State Drive), a hard disk drive, a CD (Compact Disc), a DVD (Digital Video Disc), a BD (Blu-ray Disc), a memory stick, or any appropriate combination thereof.

### Example GUI

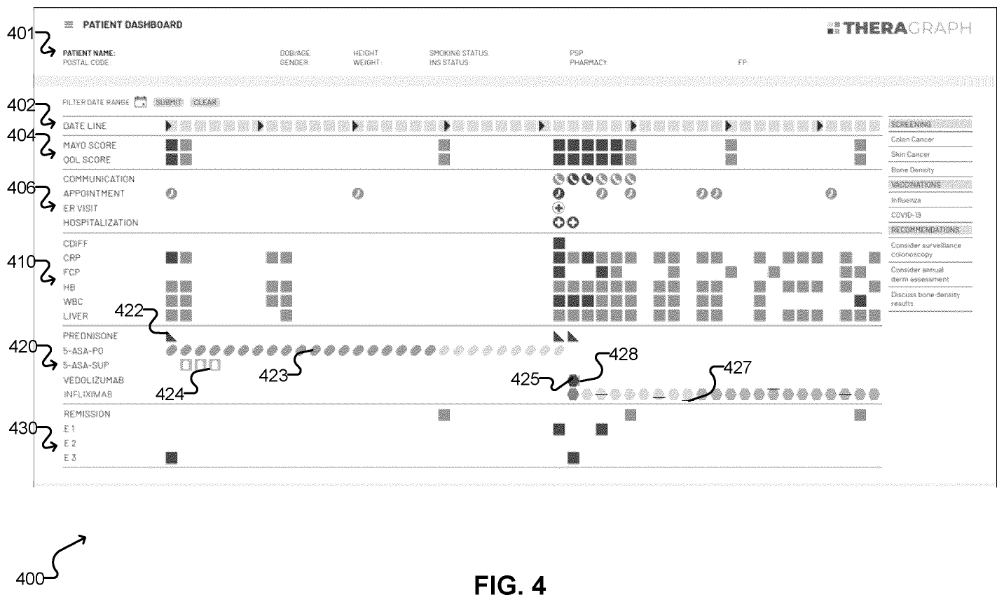

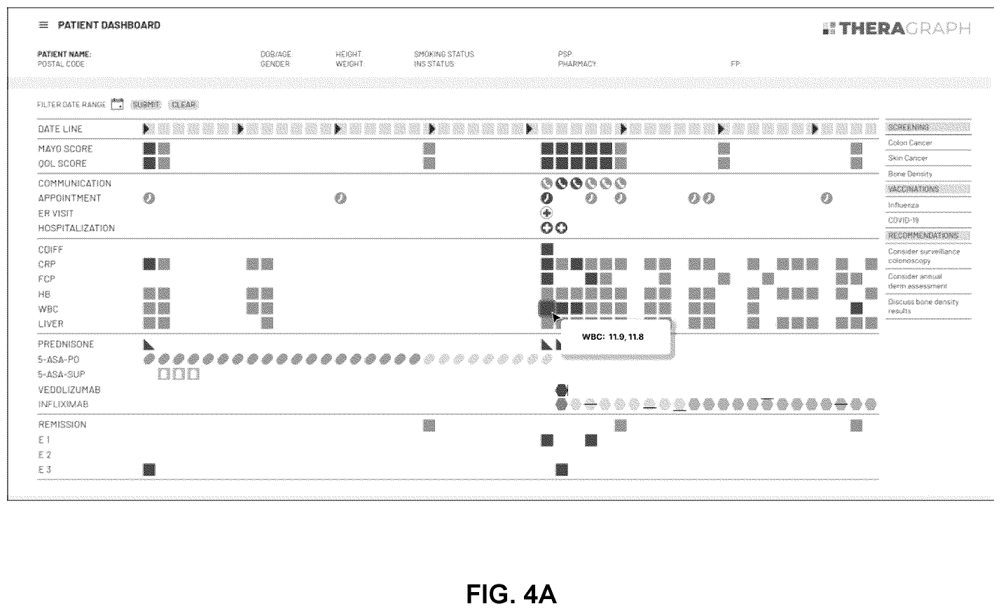

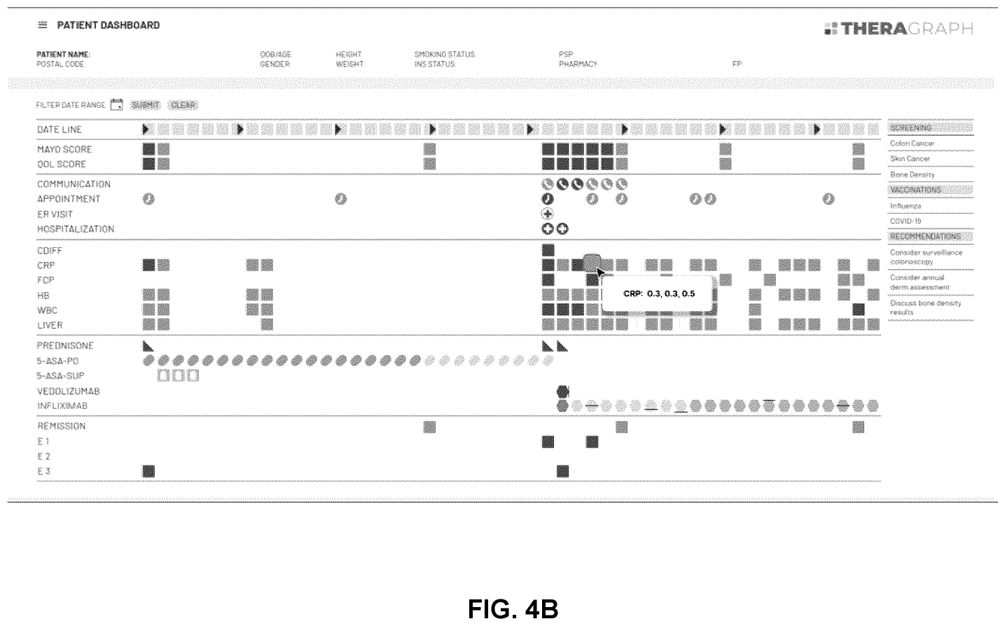

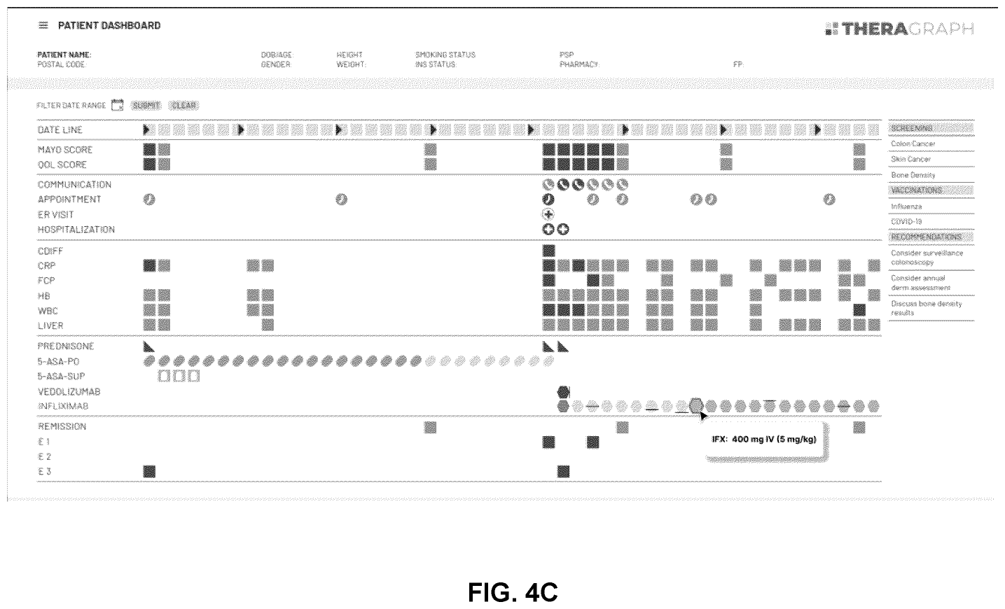

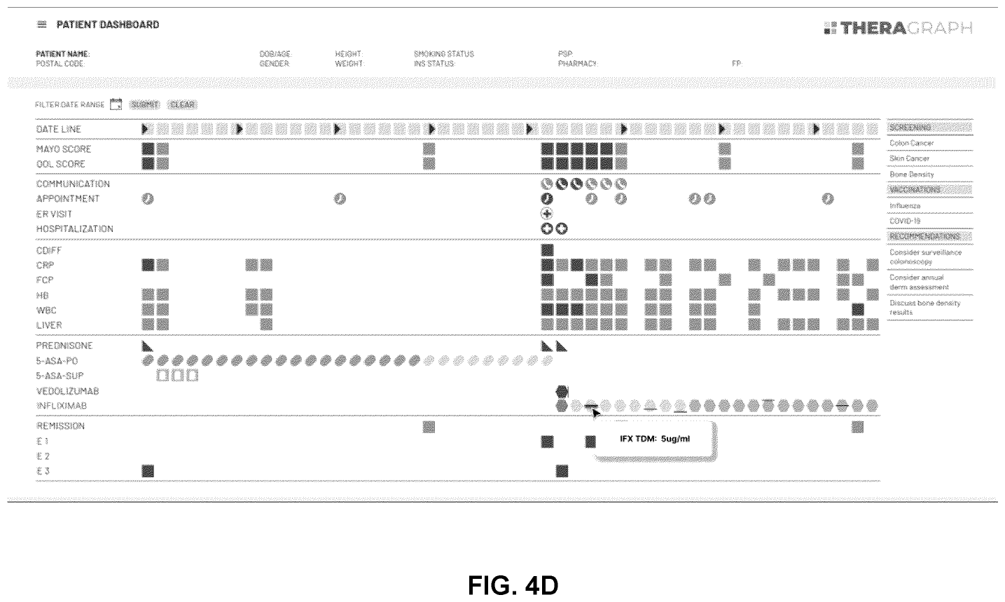

Referring now to FIG. 4, shown is an example user interface 400 having a discretized representation of patient variables over time. It is to be understood that this user interface is very specific and is provided for exemplary purposes.

User interface 400 comprises patient identifying information 401, a timeline 402 and a plurality of patient details 404-430 displayed below the timeline, which in the illustrated example comprise composite score patient variables 404 (e.g. a Mayo score and a QOL score), medical visit/communication history details 406, a plurality of test result patient variables 410 (e.g. the results of blood, stool and other tests), medication history details 420, and endoscopy score variables 430. In the illustrated example, the user interface 400 is configured for display of visual indications of a plurality of variables relating to IBD, and the test result patient variables 410 include CDIFF (Clostridium Difficile) which is an infection of the colon that can worsen IBD, CRP (C-Reactive Protein) which is a blood test that looks at overall inflammation in the body, FCP (Fecal Calprotectin) which is a stool test that monitors inflammation in stool and can gives an objective number that can be followed over time to assess response (or lack of response) to therapy, HB (Hemoglobin) which can be used to determine if someone is anemic possibly due to blood loss from an inflamed bowel, WBC (White Blood Cell) count, which is another marker for inflammation in the body, and liver enzymes (e.g. AST, ALT, ALP, Bilirubin, GGT). However, different configurations are possible for different diseases, for example by displaying indications for other patient variables relevant to such diseases, as discussed above. Such additional patient variables may be displayed instead of, or in addition to, the patient variables relating to IBD shown in FIG. 4.

The timeline 402 is broken into a number of blocks, each representing the same period of time (in this case 8 weeks). In the illustrated embodiment, arrows are used in the timeline to indicate calendar years. For each of the blocks in the timeline 402, a visual indicator for each of the patient variables and other details is displayed. If there is no data for that time block, the relevant detail/variable is either empty or shown in light grey. For patient variables where more multiple data points are available for a block, an average value for a given patient variable is determined. If the average lies outside of a desired target range it is plotted as a red square. If it lies within the target range, it is plotted as a blue square. This allows the variable to be presented in a novel, abstracted way over time. As a result of this method of displaying patient data, large numbers of variables can be displayed comfortably on the same graphic user interface. Each of the red and blue squares in the user interface 400 is also interactive, such that a user can see the underlying data points by hovering over or clicking on the square. For example, FIG. 4A is a screenshot of user interface 400 with a user overing over a red block, which causes a pop up window to appear with the relevant data therein (in this case, white blood cell counts of 11.9 and 11.8). Similarly, FIG. 4B shows a pop up window displaying data from a blue square (in this case, C-Reactive Protein scores of 0.3, 0.3 and 0.5).

In some embodiments, within the medication history details 420, different symbols are used for displaying different types of medications and delivery methods. For example, in the FIG. 4 embodiment an isosceles right-angle triangle 422 is used to indicate a tapering dosage regime, an oval 423 is used to indicate medications delivered in pill form (with darker-coloured ovals representing higher dosages and lighter-coloured ovals representing lower dosages), a rectangular symbol 424 is used to indicate medications delivered by suppository, and a hexagonal symbol 425 is used to indicate medications delivered by infusion or intravenously. In some embodiments a different symbol (e.g. an equilateral triangle pointing downward) may be used to indicate medications delivered by injection. Any of the medication symbols can be hovered over or clicked on to display dosage details (see, for example, FIG. 4C which shows a pop up window indicating a 400 mg intravenous dosage of infliximab). Additionally, a horizontal line 427 may be used to indicate results for any therapeutic drug monitoring (TDM) tests conducted for any of the medications, with the level of the line relative to the relevant symbol indicating where the concentration of the medication in the patient's blood is relative to a target concentration (i.e., if the line is at the top of the symbol, the concentration was too high, if the line is at the bottom of the symbol the concentration was too low, and if the line is in the middle of the symbol the concentration was in the target range), and the user can hover over or click on the horizontal line 427 to display the relevant measurement(s) (see, for example, FIG. 4D which shows a pop up window indicating a measured concentration of 5 μg/ml of infliximab in the patient's bloodstream). A vertical line 428 may be used to indicate a given medication was discontinued for the patient, and the user can hover over or click on the vertical line 428 to display the reason why treatment with that medication was stopped.

Methods and systems for displaying patient data according to the present disclosure allow for a logical, intuitive display of an entire patient history at a level a layperson can readily grasp, and also allow a medical professional to see, at a glance, a patient's entire history, which would otherwise require them to review a large volume of materials. The display of patient variables in such a configuration also allows them to be easily viewed in relationship to other elements of patient management, such as medication use.

Referring back to FIG. 1, the user interface consistent with an orthodox clinical dashboard according to the prior art (e.g. Swedish patient registry) for patients with inflammatory bowel disease. As can be seen, only one variable (i.e. F-Calprotectin) is plotted in relation to other clinical parameters. It is plotted in a form of a line graph. As could be imagined, plotting many relevant clinical variables at the same time would be visually overwhelming. However, the user interface of FIG. 4 enables concurrent display of many relevant clinical variables with ease, because they are not displayed as line graphs or lists of numbers, but rather in bimodal representation.

The embodiments of the systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Each program may be implemented in a high level procedural or object oriented programming or scripting language, or both, to communicate with a computer system. However, alternatively the programs may be implemented in assembly or machine language, if desired. In any case, the language may be a compiled or interpreted language. Each such computer program may be stored on a storage media or a device (e.g. ROM or magnetic diskette), readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product including a physical non-transitory computer readable medium that bears computer usable instructions for one or more processors. The medium may be provided in various forms, including one or more diskettes, compact disks, tapes, chips, magnetic and electronic storage media, and the like. The computer useable instructions may also be in various forms, including compiled and non-compiled code.

Throughout the foregoing discussion, numerous references may be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments of the present disclosure may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

It will be appreciated that numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing implementation of the various example embodiments described herein.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As will be apparent to those skilled in the art in light of the foregoing disclosure, many alterations and modifications are possible to the methods and systems described herein. While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as may reasonably be inferred by one skilled in the art. The scope of the claims should not be limited by the embodiments set forth in the examples, but should be given the broadest interpretation consistent with the foregoing disclosure.

Numerous modifications and variations of the present disclosure are possible in light of the above teachings. It is therefore to be understood that within the scope of the appended claims, the disclosure may be practised otherwise than as specifically described herein.

1. A method comprising:
maintaining a framework for categorizing time series patient data into one of a plurality of possible categories;
receiving time series patient data spanning over a total time period, the time series patient data comprising a plurality of patient variables over time, and the total time period covering an entire patient history;
dividing the total time period into a plurality of time intervals;
for each patient variable for each time interval of the plurality of time intervals, analyzing the time series patient data of the time interval using the framework to produce a category of the plurality of possible categories;
generating graphic data for a GUI (Graphic User Interface) to display, for each patient variable for each time interval, a visual indication of the category for the time interval; and
generating the GUI or transmitting the graphic data.

2. The method of claim 1, wherein the plurality of possible categories consists of two categories, such for each time interval, the visual indication for each patient variable is a bimodal representation.

3. The method of claim 2, wherein the framework comprises a threshold function, such that for each patient variable, and for each time interval, an average value of the patient variable over the time interval is compared to a threshold value or a target range to determine the bimodal representation for the patient variable in the time interval.

4. The method of claim 2, wherein the bimodal representation for each patient variable comprises a first color for acceptable value of the patient variable and a second color for unacceptable value of the patient variable.

5. The method of claim 4, wherein the first color is blue and a second color is red.

6. The method of claim 4, further comprising providing a visual representation in a third color for any time interval for which there is no data for the patient variable.

7. The method of claim 6, wherein the patient variables comprise CDIFF (Clostridium Difficile), CRP (C-Reactive Protein), FCP (Fecal Calprotectin), HB (Hemoglobin), WBC (White Blood Cell) count, and liver enzymes.

8. The method of any claim 7, wherein the patient variables comprise fasting plasma glucose, glycated hemoglobin (HbA1c), random plasma glucose, blood pressure, lipid profile, creatinine and estimated glomerular filtration rate (eGFR), urine albumin-to-creatinine ratio, BMI, waist circumference, serum insulin levels, C-peptide levels, liver function test, thyroid function tests, vitamin D levels, hemoglobin level, and microalbuminuria.

9. The method of claim 8, wherein the patient variables comprise psoriasis area severity index (PASI), body surface area affected (BSA), dermatology life quality index (DLQI), itch severity scale (ISS), visual analog scale for pain (VAS), nail psoriasis severity index (NAPSI), physician global assessment (PGA), patient global assessment (PtGA), Health related quality of life (HRQOL), psoriasis symptom index (PSI), joint involvement, serum drug levels, and adverse psoriasis events.

10. The method of claim 9, wherein the patient variables comprise eczema area and severity index (EASI) score, scoring atopic dermatitis (SCORAD) index, investigator global assessment (IGA), patient oriented eczema measure (POEM), visual analog scale (VAS) for itch, patient global assessment (PtGA), dermatology life quality index (DLQI), CBC, liver function tests, renal function tests, serum drug levels, skin barrier function (transepidermal water loss/skin hydration), and adverse atopic dermatitis events.

11. The method of claim 10, wherein the patient variables comprise hydradenitis suppurativa clinical response (HiSCR), investigator global assessment (IGA), patient global assessment (PtGA), pain, abscess, inflammatory nodules, drainage/fistulas, dermatology quality of life index (DLQI), hydradenitis suppurativa quality of life (HiSQL), CBC, liver function tests, renal function tests, serum drug levels, and adverse hydradenitis suppurativa events.

12. The method of claim 11, wherein the patient variables comprise American College of Rheumatology (ACR) response criteria, Disease Activity Score (DAS), Health Assessment Questionnaire Disability Index (HAQ-DI), C-reactive protein (CRP) level, Erythrocyte sedimentation rate (ESR), Physician's Global Assessment of Disease Activity, Patient's Global Assessment of Disease Activity, Visual Analog Scale (VAS) pain, Swollen joint count, tender joint count, Radiographic joint damage, Short Form 36 (SF-36), EuroQol-5 Dimension (EQ-5D), Fatigue, Morning stiffness duration, Patient's assessment of joint pain, Patient's assessment of joint stiffness, Rheumatoid Arthritis Quality of Life (RAQOL) questionnaire, serum levels of the biologic drug being studied, adverse rheumatoid arthritis events.

13. The method of claim 12, wherein the patient variables comprise American College of Rheumatology (ACR) response criteria, Psoriasis Area and Severity Index (PASI), Disease Activity Score (DAS), Health Assessment Questionnaire Disability Index (HAQ-DI), C-reactive protein (CRP) level, Erythrocyte sedimentation rate (ESR), Physician's Global Assessment of Disease Activity, Patient's Global Assessment of Disease Activity, Visual Analog Scale (VAS) pain, Swollen joint count, Tender joint count, Radiographic joint damage, Short Form 36 (SF-36), EuroQol-5 Dimension (EQ-5D), Fatigue, Morning stiffness duration, Patient's assessment of joint pain, Patient's assessment of joint stiffness, Psoriatic Arthritis Quality of Life (PsAQoL) questionnaire, and adverse psoriatic arthritis events.

14. The method of claim 13, wherein the patient variables comprise Assessment of SpondyloArthritis international Society (ASAS) criteria, Bath Ankylosing Spondylitis Disease Activity Index (BASDAI), Bath Ankylosing Spondylitis Functional Index (BASFI), Bath Ankylosing Spondylitis Metrology Index (BASMI), C-reactive protein (CRP) level, Erythrocyte sedimentation rate (ESR), Physician's Global Assessment of Disease Activity, Patient's Global Assessment of Disease Activity, Visual Analog Scale (VAS) pain, Spinal pain, Spinal mobility, Short Form 36 (SF-36), EuroQol-5 Dimension (EQ-5D), Fatigue, Sleep disturbances, Patient's assessment of joint pain, Patient's assessment of joint stiffness, Ankylosing Spondylitis Quality of Life (ASQoL) questionnaire, and adverse Ankylosing Spondylitis events.

15. The method of claim 14, wherein the patient variables comprise Forced expiratory volume in 1 second (FEV1), Peak expiratory flow rate (PEFR), Asthma Control Questionnaire (ACQ), Asthma Quality of Life Questionnaire (AQLQ), Asthma Symptom Utility Index (ASUI), Asthma exacerbation rate, Rescue medication use, Fractional exhaled nitric oxide (FeNO), Blood eosinophil count, Total IgE levels, Forced vital capacity (FVC), Bronchodilator reversibility, Allergen-specific IgE levels, Pulmonary function tests (PFTs, which measure lung function, including FEV1, FVC, and other parameters), Patient-reported outcomes (PROs), Inflammatory cytokine levels, Asthma exacerbation severity, Asthma symptom score, Asthma control status, which measures the level of asthma control based on established criteria, such as the Global Initiative for Asthma (GINA) guidelines, and adverse asthma events.

16. The method of claim 1, wherein each time interval is at least six weeks.

17. The method of claim 1, wherein each time interval is eight weeks.

18. A method for execution by a server, comprising:
maintaining a framework for categorizing time series patient data into one of a plurality of possible categories useful for managing patients with one or more chronic health conditions;
receiving, from a first client computing device, time series patient data spanning over a total time period, the time series patient data comprising a plurality of patient variables over time, and the total time period covering an entire patient history;
dividing the total time period into a plurality of time intervals;
for each patient variable for each time interval of the plurality of time intervals, analyzing the time series patient data of the time interval using the framework to produce a category of the plurality of possible categories;
generating graphic data for a GUI (Graphic User Interface) to display, for each patient variable for each time interval, a visual indication of the category for the time interval; and
transmitting, to a second client computing device, the graphic data from which the second client computing device can generate the GUI.

19. The method of claim 18, wherein the plurality of possible categories consists of two categories, such for each time interval, the visual indication for each patient variable is a bimodal representation.

20. The method of claim 19, wherein the framework comprises a threshold function, such that for each patient variable, and for each time interval, an average value of the patient variable over the time interval is compared to a threshold value to determine the bimodal representation for the patient variable in the time interval.

21. The method of claim 19, wherein the bimodal representation for each patient variable comprises a first color for acceptable value of the patient variable and a second color for unacceptable value of the patient variable.

22. The method of claim 21, wherein the first color is blue and a second color is red.

23. The method of claim 21, further comprising providing a visual representation in a third color for any time interval for which there is no data for the patient variable.

24. The method of claim 18, wherein the one or more chronic health conditions include IBD (Inflammatory Bowel Disease), and wherein the patient variables comprise at least some of CDIFF (Clostridium Difficile), CRP (C-Reactive Protein), FCP (Fecal Calprotectin), HB (Hemoglobin), WBC (White Blood Cell) count, and liver enzymes.

25. The method of claim 24, wherein the one or more chronic health conditions include diabetes, and wherein the patient variables comprise fasting plasma glucose, glycated hemoglobin (HbA1c), random plasma glucose, blood pressure, lipid profile, creatinine and estimated glomerular filtration rate (eGFR), urine albumin-to-creatinine ratio, BMI, waist circumference, serum insulin levels, C-peptide levels, liver function test, thyroid function tests, vitamin D levels, hemoglobin level, and microalbuminuria.

26. The method of claim 25, wherein the one or more chronic health conditions include psoriasis, and wherein the patient variables comprise psoriasis area severity index (PASI), body surface area affected (BSA), dermatology life quality index (DLQI), itch severity scale (ISS), visual analog scale for pain (VAS), nail psoriasis severity index (NAPSI), physician global assessment (PGA), patient global assessment (PtGA), Health related quality of life (HRQOL), psoriasis symptom index (PSI), joint involvement, serum drug levels, and adverse psoriasis events.

27. The method of claim 26, wherein the one or more chronic health conditions include atopic dermatitis, and wherein the patient variables comprise eczema area and severity index (EASI) score, scoring atopic dermatitis (SCORAD) index, investigator global assessment (IGA), patient oriented eczema measure (POEM), visual analog scale (VAS) for itch, patient global assessment (PtGA), dermatology life quality index (DLQI), CBC, liver function tests, renal function tests, serum drug levels, skin barrier function (transepidermal water loss/skin hydration), and adverse atopic dermatitis events.

28. The method of claim 27, wherein the one or more chronic health conditions include hydradenitis suppurativa, and wherein the patient variables comprise hydradenitis suppurativa clinical response (HiSCR), investigator global assessment (IGA), patient global assessment (PtGA), pain, abscess, inflammatory nodules, drainage/fistulas, dermatology quality of life index (DLQI), hydradenitis suppurativa quality of life (HiSQoL), CBC, liver function tests, renal function tests, serum drug levels, and adverse hydradenitis suppurativa events.

29. The method of claim 28, wherein the one or more chronic health conditions include rheumatoid arthritis, and wherein the patient variables comprise American College of Rheumatology (ACR) response criteria, Disease Activity Score (DAS), Health Assessment Questionnaire Disability Index (HAQ-DI), C-reactive protein (CRP) level, Erythrocyte sedimentation rate (ESR), Physician's Global Assessment of Disease Activity, Patient's Global Assessment of Disease Activity, Visual Analog Scale (VAS) pain, Swollen joint count, tender joint count, Radiographic joint damage, Short Form 36 (SF-36), EuroQol-5 Dimension (EQ-5D), Fatigue, Morning stiffness duration, Patient's assessment of joint pain, Patient's assessment of joint stiffness, Rheumatoid Arthritis Quality of Life (RAQOL) questionnaire, serum levels of the biologic drug being studied, adverse rheumatoid arthritis events.

30. The method of claim 29, wherein the one or more chronic health conditions include psoriatic arthritis, and wherein the patient variables comprise American College of Rheumatology (ACR) response criteria, Psoriasis Area and Severity Index (PASI), Disease Activity Score (DAS), Health Assessment Questionnaire Disability Index (HAQ-DI), C-reactive protein (CRP) level, Erythrocyte sedimentation rate (ESR), Physician's Global Assessment of Disease Activity, Patient's Global Assessment of Disease Activity, Visual Analog Scale (VAS) pain, Swollen joint count, Tender joint count, Radiographic joint damage, Short Form 36 (SF-36), EuroQol-5 Dimension (EQ-5D), Fatigue, Morning stiffness duration, Patient's assessment of joint pain, Patient's assessment of joint stiffness, Psoriatic Arthritis Quality of Life (PsAQoL) questionnaire, and adverse psoriatic arthritis events.

31. The method of claim 30, wherein the one or more chronic health conditions include Ankylosing Spondylitis, and wherein the patient variables comprise Assessment of SpondyloArthritis international Society (ASAS) criteria, Bath Ankylosing Spondylitis Disease Activity Index (BASDAI), Bath Ankylosing Spondylitis Functional Index (BASFI), Bath Ankylosing Spondylitis Metrology Index (BASMI), C-reactive protein (CRP) level, Erythrocyte sedimentation rate (ESR), Physician's Global Assessment of Disease Activity, Patient's Global Assessment of Disease Activity, Visual Analog Scale (VAS) pain, Spinal pain, Spinal mobility, Short Form 36 (SF-36), EuroQol-5 Dimension (EQ-5D), Fatigue, Sleep disturbances, Patient's assessment of joint pain, Patient's assessment of joint stiffness, Ankylosing Spondylitis Quality of Life (ASQoL) questionnaire, and adverse Ankylosing Spondylitis events.

32. The method of claim 31, wherein the one or more chronic health conditions include asthma, and wherein the patient variables comprise Forced expiratory volume in 1 second (FEV1), Peak expiratory flow rate (PEFR), Asthma Control Questionnaire (ACQ), Asthma Quality of Life Questionnaire (AQLQ), Asthma Symptom Utility Index (ASUI), Asthma exacerbation rate, Rescue medication use, Fractional exhaled nitric oxide (FeNO), Blood eosinophil count, Total IgE levels, Forced vital capacity (FVC), Bronchodilator reversibility, Allergen-specific IgE levels, Pulmonary function tests (PFTs, which measure lung function, including FEV1, FVC, and other parameters), Patient-reported outcomes (PROs), Inflammatory cytokine levels, Asthma exacerbation severity, Asthma symptom score, Asthma control status, which measures the level of asthma control based on established criteria, such as the Global Initiative for Asthma (GINA) guidelines, and adverse asthma events.

33. The method of claim 18, wherein each time interval is at least six weeks.

34. The method of claim 18, wherein each time interval is eight weeks.

35. The method of claim 18, wherein the first client computing device is different from the second client computing device.

36. The method of claim 18, wherein the first client computing device is same as the second client computing device.

37. A non-transitory computer readable medium having recorded thereon statements and instructions that, when executed by a processor of a server, configure the server to implement the method of claim 18.

38. A server, comprising:
a network adapter; and
variable categorization circuitry coupled to the network adapter and configured to implement the method of claim 18.

39. The server of claim 38, wherein:
the variable categorization circuitry comprises a processor; and
the server further comprises a non-transitory computer readable medium having recorded thereon statements and instructions that, when executed by the processor, configures the processor as the variable categorization circuitry.

40. The server of claim 39, wherein the non-transitory computer readable medium stores the framework for categorizing time series patient data.
